(19) United States
(12) Patent Application Publication
(10) Pub. No.: US 2024/0282415 A1
(43) Pub. Date: Aug. 22, 2024

(54) SYSTEMS AND METHODS FOR PROVIDING ELECTRONIC AND NON-ELECTRONIC RECORDS

(71) Applicant: Optum, Inc., Minnetonka, MN (US)

(72) Inventors: Marlene McIntyre, Suwanee, GA (US); James Bridges, Chapel Hill, NC (US); Jean Gibson, Bristol, VT (US); Brent O'Dell, Murfreesboro, TN (US)

(21) Appl. No.: 18/170,582

(22) Filed: Feb. 17, 2023

(51) Int. Cl. G16H 10/60; G06Q 40/08; G16H 40/20

(52) U.S. Cl. CPC G16H 10/60; G06Q 40/08; G16H 40/20

(57) ABSTRACT

In an embodiment, systems and methods for retrieving medical records are provided. A request for medical records is received from a payor by a records system. The request may include a list of the medical providers and criteria. The criteria may specify whether the payor only wants electronic records or both electronic and non-electronic records. The records system compares the list of providers with a list of providers who use electronic records and determines which providers of the request use electronic records and which providers of the request do not use electronic records. For each medical provider of the request that uses electronic records, the records system may request the records from each provider using an electronic records workflow. For each medical provider of the request that does not use electronic records, the records system may request the records from each provider using a non-electronic request workflow.

### BACKGROUND

Payors, such as insurance payors, may request charts or medical records from medical providers. These medical records may be used for a variety of purposes such as risk adjustment, claims auditing and validation, and for determining that patients are receiving the correct standard of care from the medical providers.

However, requesting and receiving the medical records from the medical providers can be a difficult task. First, each payor may have a particular format that they want the medical records to be provided in. Second, each medical provider may store medical records in some combination of electronic and non-electronic records, making it difficult to determine how to retrieve the desired medical records. Third, each medical provider may have a different process or procedure for payors to use when requesting records.

### SUMMARY

In an embodiment, systems and methods for retrieving medical records are provided. A request for medical records is received from a payor by a records system. The request may include a list of the medical providers to provide medical records, and criteria. The criteria may specify the patient(s) whose records are being sought, one or more of a use case for the records, a date when the records should be completed, whether the payor only wants electronic records or both electronic and non-electronic records, and a desired format for the records. The records system compares the list of medical providers with a list of medical providers active in the records system and determines which medical providers of the request are active in the records system and which medical providers of the request are not active in the records system. Based on the criteria and list of providers the records system may select a subset of providers that are capable of fulfilling the request. For each medical provider in the subset that uses electronic records, the records system may request the records from each provider using an electronic records workflow. For each medical provider of the subset that is not active in the records system, or for whom the electronic records workflow has failed, the records system may request the records from each provider using a non-electronic request workflow. The records, both electronic and non-electronic, may be provided to the requesting payor as they are received and according to the criteria included in the request.

The systems and method for providing records described herein have many advantages over the prior art. First, by maintaining a list of providers who are active in the electric records system, when a request for records is received from a payor, the portions of the request that can be fulfilled electronically and the portions of the request that cannot be fulfilled electronically can be quickly determined. The payor may then be informed regarding how long the request will likely take to be fulfilled based on the split between electronic and non-electronic records. In addition, depending on the use case for the records, the request may be automatically modified so that it only returns electronic records, which will result in a faster completion of the request. Third, by maintaining information about the procedures each provider uses for maintaining and providing electronic and non-electronic records, and using those procedures to request records from providers, payors are able to request records using a single request and without knowledge about how each provider expects their requests to be formatted and submitted.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate systems and methods for providing electronic and non-electronic records. Together with the description, the figures further serve to explain the principles of the systems and methods for providing electronic and non-electronic records described herein and thereby enable a person skilled in the pertinent art to make and use the systems and methods for providing electronic and non-electronic records.

FIG. 1 is an example environment for providing electronic and non-electronic records;

FIG. 2 is an illustration of an example method for providing electronic and non-electronic records;

FIG. 3 is an illustration of an example method for performing an electronic record workflow;

FIG. 4 is an illustration of an example method for performing a non-electronic record workflow; and

FIG. 5 shows an example computing environment in which example embodiments and aspects may be implemented.

### DETAILED DESCRIPTION

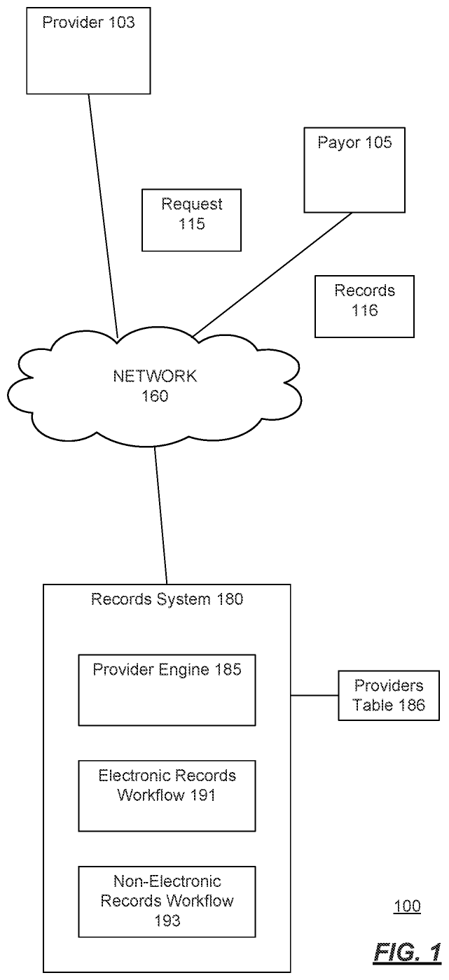

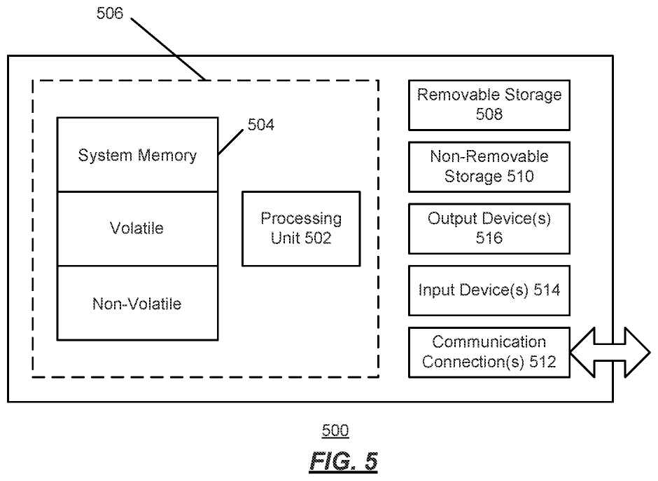

FIG. 1 is an example environment 100 for providing electronic and non-electronic records. As shown, the environment 100 may include one or more providers 103 and one or more payors 105, in communication with a records system 180 through a network 160. The network 160 may include a combination of private networks (e.g., LANs) and public networks (e.g., the Internet). Each of the providers 103, the payors 105, and the records system 180 may use, or may be partially implemented by, one or more general purpose computing devices such as the computing device 500 illustrated in FIG. 5.

The payors 105 may include insurance companies who pay claims for patients treated by the providers 103. The providers 103 may include medical providers 103 such as hospitals, clinics, and physician practices.

As described above, payors 105 may have to periodically request medical records 116 from medical providers 103. The requested medical records may be associated with patients covered by the payors 105 and may be used by the payors 105 for a variety of purposes such as risk adjustment and quality programs, to ensure appropriateness of provider coding and documentation, the completeness and quality of care provided, and to support care management and other care related programs. In order to help payors 105 request records 116 and to ensure the timely delivery of both electronic and non-electronic records 116, the environment 100 includes the records system 180. As shown, the records system 180 includes several components, including but not limited to, a provider engine 185, an electronic records workflow 191, and a non-electronic records workflow 193. Each of the components of the records system 180 may be implemented together or separately using one or more general purpose computing devices such as the computing device 500 illustrated further with respect to FIG. 5.

The provider engine 185 may maintain data about each of the providers 103 in a data structure such as the providers table 186. The providers table 186 may include an entry for each provider 103 along with a provider identifier that uniquely identifies each provider 103. In the addition, each entry may indicate whether the provider is active in the records system 180. As used herein, a provider is active in the records system 180 if they accept electronic records requests from the records system 180.

Further, each entry in the providers table 186 may specify the earliest date that electronic records 116 are available, may specify what format(s) the records 116 are available in, and may specify how the electronic records 116 may be requested from the provider 103. For example, the entry for a medical provider 103 may include an address where requests for records 116 can be submitted or may indicate an API that can be used to request records 116 from the provider 103.

In some embodiments, for providers 103 that are not active in the records system 180, the entry in the providers table 186 for the provider 103 may include information on how an appointment to collect the non-electronic records 116 can be made with the provider 103. Other information may be included in the providers table 186.

The records system 180 may receive a request 115 for records 116 from a payor 105. The request 115 may include information about the records 116 that the payor 105 is requesting such as an identifier of the provider 103 whose records 116 the payor 105 is requesting, date ranges associated with the request 116, and identifiers of particular patients or members. In some embodiments, the provider identifiers may include a tax identifier of the provider 103, or an identifier assigned to the provider 103 by the records system 180.

In some embodiments, the request 115 may further include criteria about the request 115. The criteria may include information about how the payor 105 would like the request 115 fulfilled. Example criteria includes whether or not the payor wants electronic records 116 only or some combination of non-electronic records 116 and electronic records 116, a due date for the request 115, and the format or formats that the payor 105 would like the records 116 (e.g., PDF format or native format). The criteria may further indicate whether the payor 105 would like all of the requested records 116 in one delivery, or if they would like to receive the records 116 on a rolling basis as they are retrieved by the records system 180. Other types of criteria may be included in the request 115

In some embodiments, the criteria may include an intended use case for the requested medical records 116. The use cases may include risk adjustment, quality/HEDIS programs, care coordination, care management, and payment accuracy. Based on the intended use case, the records system 180 may determine if the request 115 should be fulfilled using electronic records 116 only or whether a combination of electronic records 116 and non-electronic records 116 may be used.

A payor 105 may provide the request 115 to the records system 180. In some embodiments, the request 115 may be an electronic request 115 and may be provided by the payor 105 to the records system 180 through the network 160 using e-mail or a graphical user interface provided by the records system 180 for inputting requests 115 including criteria. In other embodiments, a first representative associated with the payor 105 may call a second representative associated with the records system 180, and the second representative may create a request 115 for the payor 105 based on information provided by the first representative including criteria. Other methods may be used.

Upon receiving the request 115, the records system 180 may begin the process of fulfilling the request 115. As a first step, the records system 180 may extract the provider identifiers from the request 115 and may determine which of the identified providers 103 are active in the records system 180, and which are not. In some embodiments, the records system 180 may determine the providers 103 that are active in the records system 180 using the information in the providers table 186.

For example, a payor 105 may generate and send a request 115 for records 116 dated between 2020-2022 from a first provider 103, a second provider 103, and a third provider 103. The records system 180 may cross reference provider identifiers of the first provider 103, the second provider 103, and the third provider 103 with the providers table 186 and may determine that the first and second provider 103 are active in the records system 180 and that the third provider 103 is not active in the records system 180.

Based on the determination of which providers 103 are active in the records system 180, the records system 180 may determine which portions of the request 115 to process using the electronics records workflow 191 or the non-electronic workflow 193. Continuing the example above, the records system 180 may process the portions of the request 115 directed to the first and second providers 103 using the electronic records workflow 191 and may process the portion of the request 115 directed to the third provider 103 using the non-electronic records workflow 193.

How the request 115 is processed may also depend on the criteria associated with the request 115. Where the criteria indicates that only electronic records 116 should be considered, the records system 180 should only process requests 115 using the electronic records workflow 191 and should otherwise disregard portions of the request 115 that cannot be fulfilled electronically. Depending on the embodiment, where some portions of the request 115 cannot be fulfilled electronically, the records system 180 may send a message or may provide an indication to the payor 105 that some portions of the request 115 will not be fulfilled.

Continuing the example above, the records system 180 may process the portions of the request 115 directed to the first and second providers 103 using the electronic records workflow 191 and may discard the portion of the request 115 directed to the third provider 103 because it is not active in the records system 180 and its records cannot be retrieved using the electronic records workflow 191.

Returning to the criteria, where a due date is included in the criteria of the request 115, the records system 180 may consider whether a request 115 can be fulfilled completely within the time frame defined by the due date before processing the request 115. Portions of the request 115 that can be completed by the due date may be processed by the provider engine 185, while those portions of the request 115 that cannot be fulfilled by the due date may be discarded.

Continuing the example above, the due date specified in the criteria may be two weeks from the date that the request 115 was submitted. Based on information in the providers table 186, the records system 180 may determine that the portions of the request 115 directed to the first and second providers 103 are likely to be fulfilled within two weeks, but the portion of the request 115 directed to the third provider 103 is unlikely to be fulfilled within two weeks. Accordingly, the records system 180 may process the portions of the request 115 directed to the first and second providers 103 and may discard the portions of the request directed to the third provider 103.

In some embodiments, the processing of the request 115 may also determine on the date ranges associated with the request 115. Because some providers 103 may only support electronic records after a certain date, the records system 180 may use the date ranges associated with the request 115 to determine if some portions of a request 115 for a provider 103 should be processed using the electronics records workflow 191 and other portions should be processed using the non-electronic records workflow 193.

Continuing the example above, the request 115 for records from the first provider 103, the second provider 103, and the third provider 103 may have date ranges of 2020-2022. The providers table 186 may indicate that the first provider 103 has electronic records since 2005, that the second provider 103 has electronic records since 2021, and that the third provider 103 is not active in the records system 180. Accordingly, the records system 180 may process the portions of the request 115 directed to the first provider 103 using the electronic records workflow 191, may process the portions of the request 115 directed to the second provider 103 with dates that are after 2001 using the electronic records workflow 191, may process the portions of the request 115 directed to the second provider 103 with dates that are before 2001 using the non-electronic records workflow 193, and may process the portions of the request 115 directed to the third provider 103 using the non-electronic records workflow 193.

The electronic records workflow 191 may be a process that the records system 180 follows to retrieve electronic records 116 from a provider 103 identified in a request 115 according to the criteria. As a first step, the provider engine 185 may retrieve information about the provider 103 from the providers table 186. The information may indicate how the records system 180 should request electronic records 116 from the provider 103. For example, the information may indicate an API, URL, or other endpoint or system that the records system 180 may use to request the electronic records 116 from the provider 103. Alternatively, the records system 180 may have direct access to the electronic records 116 by way of an agreement between the providers 103 and the records system 180.

The records system 180, as part of the electronic records workflow 191, may request the electronic records 116 from the provider 103 according to the information from the providers table 186. Where the criteria associated with the request 115 indicates that the electronic records 116 should be provided in a particular format, the records system 180 may request that the provider 103 provide the electronic records 116 in the requested format.

Continuing the electronics workflow 191, the records system 180 may receive the electronic records 116 from the provider 103. If the records 116 are in the format specified in the criteria of the request 115, the records system 180 may provide the electronic records 116 to the provider 103. Otherwise, the records system 180 may convert the electronic records 116 to the format specified in the request 115.

In some embodiments, a provider 103 may determine that some of the requested records 116 are not available in an electronic form, or that the electronic workflow 191 has failed or could not be completed with respect to some of the requested records 116. The provider 103 may indicate the records 116 to the records system 180, and the records system 180 may request non-electronic versions of the unavailable records 116 from the provider 103 using the non-electronic records workflow 193.

As described above, each request 115 may include multiple providers 103. Accordingly, rather than provide the electronic records 116 from each provider 103 to the payor 105 as they are received, in some embodiments, the records system 180 may wait until all or most of the records 116 are received before providing the records 116 to the payor 105. Whether or not the records system 180 sends records 116 to the payor 105 as they are received, or in one bulk transaction, may be set by the payor 105 in the criteria of the request 115.

The non-electronic records workflow 193 may be a process that the records system 180 follows to retrieve non-electronic records 116 from a provider 103 identified in a request 115 according to the criteria. As a first step, the provider engine 185 may retrieve information about the provider 103 from the providers table 186. The information may indicate how the records system 180 may contact the provider 103 to set up an appointment to retrieve copies of the records 116 indicated by the request 115.

For example, the information may indicate a phone number, email address, or other means that a representative of the records system 180 may use to set up the appointment. As another example, the information may include a form that the representative may use to set up the appointment. The form may indicate the records 116 specified in the request 115. The form may then be faxed or emailed to the provider 103 at an address or number indicated by the information.

An appointment as used herein may include an appointment for a representative of the records system 180 to travel to a location where the non-electronic records 116 are available to be collected or copied by the representative. The appointment may further include an appointment for a representative of the provider 103 to collect or make copies of the records 116 of behalf of the records system 180.

After receiving the non-electronic records, either directly from the provider 103 or as collected by the representative of the records system 180, the records system 180 may convert the non-electronics records 116 into an electronic format specified by the criteria associated with the request 115. Depending on the embodiment, the records system 180 may provide the records 116 to the payor 105 as they are received by the records system 180, or the records system 180 may wait until all of the electronic and non-electronic records 116 have been received to send the records 116 to the payor 105.

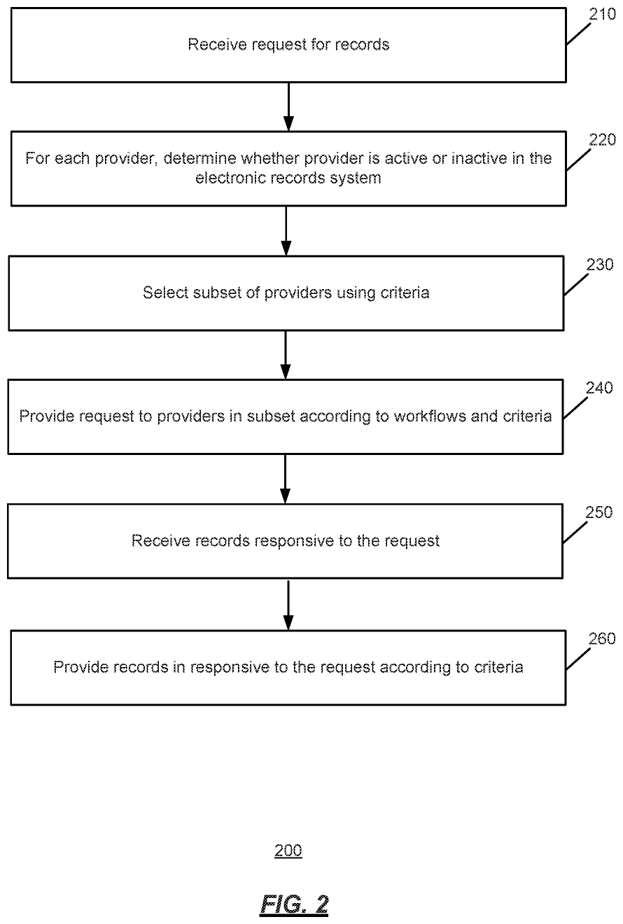

FIG. 2 is an illustration of an example method for providing electronic and non-electronic records. The method 200 may be implemented by the records system 180.

At 210, a request for records is received. The request 115 may be received by the records system 180 from a payor 105. The request 115 may be a request for records 116. The records 116 may be medical records and may include both electronic and non-electronic records. The request 115 may identify multiple medical providers 103 that have the records 116. The request 116 may identify the types of records that the payor 105 is interested in as well as a date range. The request 115 may further be associated with criteria regarding a use for the request 115, when the payor 105 would like the records 116, and whether the payor 105 wants just electronic records 116 or both electronic and non-electronic records 116.

At 220, for each provider identified in the request 115, whether the provider is or is not active in the electronic records system is determined. The determination may be made by the records system 180 using information contained in a providers table 186. The providers table 186 may include information about each provider 103 such as whether or not the provider 103 is active in the electronic records system.

At 230, a subset of providers is selected based on the criteria. The subset of providers 103 may be selected from among the providers 103 identified in the request 115 based on the criteria by the records system 180. Where the criteria includes a use case for the records, the records system 180 may determine if the request 103 can be fulfilled using only electronic records 116 or a combination of electronic and non-electronic records 166. If the request 115 can be fulfilled using only electronic records 116, the records system 180 may select a subset of providers 103 that were determined to be active in the electronic records system. If the request 115 can be fulfilled using electronic records 116 or non-electronic records 116, the records system 180 may select a subset of providers 103 that were determined to be active or inactive in the electronic records system.

Where the criteria includes a due date, the records system 180 may select a subset of providers 103 who can fulfill the request 115 by the due date. The time a provider 103 needs to fulfil a request 115 may be specified in the providers table 186.

At 240, for each provider in the subset, the request is provided to the provider according to one or more workflows and the criteria. Where a provider 103 was determined to be active in the electronic records system, the records system 180 may provide the request 115 according to the electronic records workflow 191. Where a provider 103 was determined to not be active in the electronic records system, the records system 180 may provide the request 115 according to the non-electronic records workflow 193.

At 250, records that are responsive to the request are received. The records 116 that are responsive to the request 115 may be received by the records system 180 from the providers 103. The records 116 may include electronic records 116 or some combination or electronic and non-electronic records 116.

At 260, the records are provided in response to the request. The records 116 may be provided to the payor 105 according to the criteria associated with the request 115.

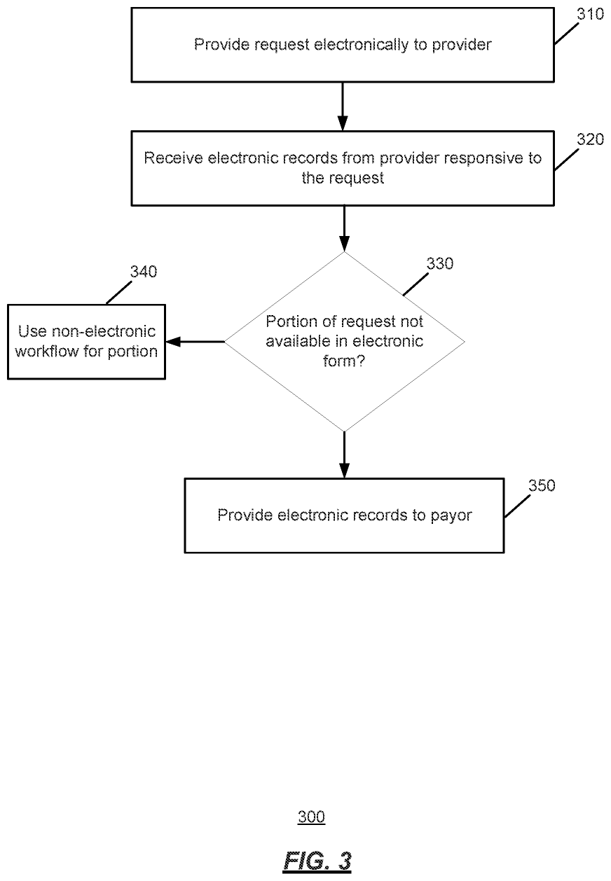

FIG. 3 is an illustration of an example method 300 for performing an electronic record workflow. The method 300 may be implemented by the records system 180.

At 310, a request is electronically provided to a provider. The request 115 may be provided to a provider 103 by the records system 180. In some embodiments, the request 115 may be provided to the provider 103 using an API or an electronic messaging system supported by the provider 103. The particular means used to provide the request 115 to the provider 103 through the network 160 may be specified in an entry in the providers table 186 associated with the provider 103. The provider 103 may be one of a plurality of providers 103 identified in the request 115 received from a payor 105.

At 320, electronic records from the provider that are responsive to the request are received. The electronic records 116 may be received by the records system 180 from the provider 103. In some embodiments, the electronic records 116 may be in format specified by the payor 105 in criteria associated with the request 115.

At 330, whether a portion of the request is not available in electronic form is determined. As may be appreciated, a particular record for a patient may not be available as an electronic record 116, even though the provider 103 indicated that the record 116 was available in the providers table 186. Depending on the embodiment, that the record was not available electronically may be determined by the provider 103 while retrieving the records 116, or may be determined by the records system 180 after inspecting the electronic records 116 that were received. If a portion of the request is not available in electronic form, the method 300 may continue to 340. Else the method 300 may continue to 350.

At 340, the missing portion of the request is requested according to the non-electronic records workflow. The missing portion may be requested by the records system 180.

At 350. The electronic records are provided. The electronic records 116 may be provided by the provider engine 185. The records 116 may be provided as soon as the electronic workflow 191 for the provider 103 is completed, or the records 116 may be provided along with other records 116 once most or all of the electronic records workflows 191 corresponding to the other providers 103 associated with the request 115 received from the payor 105 have also completed. The electronic records 116 may be provided in a format specified in the criteria associated with the request.

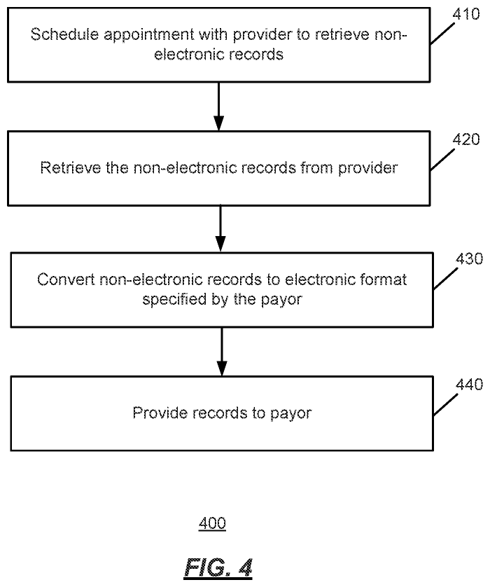

FIG. 4 is an illustration of an example method 400 for performing a non-electronic record workflow. The method 400 may be implemented by the records system 180.

At 410, an appointment with the provider to retrieve non-electronic records is scheduled. The appointment may be scheduled by the provider engine 185 using information in the providers table 186. For example, the providers table 186 may include information such as a fax number and a form that may be used to request records 116 from the provider 103.

At 420, the non-electronic records are retrieved. The electronic records 116 may be retrieved by an agent or representative of one or both of the provider 103 or the payor 105 from the provider 103. For example, the payor 105 may send a representative to the location of the provider 103 at the time of the appointment to retrieve the records 116. In some embodiments, the representative may retrieve the records 116 as digital copies.

At 430, the non-electronic records are converted to a format specified by the payor. The non-electronic records may be converted by the provider engine 185. For example, the provider engine 185 may convert the non-electronic records to the PDF format. Other formats may be used.

At 440, the records are provided to the payor. The records 116 may be provided to the payor 103 by the provider engine 185.

FIG. 5 shows an example computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 5, an example system for implementing aspects described herein includes a computing device, such as computing device 500. In its most basic configuration, computing device 500 typically includes at least one processing unit 502 and memory 504. Depending on the exact configuration and type of computing device, memory 504 may be volatile (such as random access memory (RAM)), non-volatile (such as read-only memory (ROM), flash memory, etc.), or some combination of the two. This most basic configuration is illustrated in FIG. 5 by dashed line 506.

Computing device 500 may have additional features/functionality. For example, computing device 500 may include additional storage (removable and/or non-removable) including, but not limited to, magnetic or optical disks or tape. Such additional storage is illustrated in FIG. 5 by removable storage 408 and non-removable storage 510.

Computing device 500 typically includes a variety of computer readable media. Computer readable media can be any available media that can be accessed by the device 500 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 504, removable storage 508, and non-removable storage 510 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by computing device 500. Any such computer storage media may be part of computing device 500.

Computing device 500 may contain communication connection(s) 512 that allow the device to communicate with other devices. Computing device 500 may also have input device(s) 514 such as a keyboard, mouse, pen, voice input device, touch input device, etc. Output device(s) 516 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although example implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method comprising:
receiving a request for records by a computing device from a payor, wherein the request includes a plurality of provider identifiers and criteria and further wherein each provider identifier identifies a provider of a plurality of providers;
for each provider associated with a provider identifier from the plurality of provider identifiers, determining whether the provider is active in an electronic records system by the computing device;
based on the determination of whether the providers are active in the electronic records system and the criteria, selecting a subset of the providers associated with a provider identifier of the plurality of provider identifiers by the computing device; and
providing the request to each provider in the subset according to the criteria by the computing device.

2. The method of claim 1, wherein providing the request to each provider in the subset according to the criteria comprises:
for each provider in the subset determined to be active in the electronic records system, providing the request to the provider according to an electronic records workflow and the criteria;
for each provider in the subset determined to be active in the electronic records system, providing the request to the provider according to a non-electronic records workflow and the criteria.

3. The method of claim 2, wherein providing the request to the provider according to the electronic records workflow and the criteria comprises:
providing the request electronically to the provider;
receiving from the provider electronic records responsive to the request; and
converting electronic records into a format specified by the payor in the criteria.

4. The method of claim 3, further comprising:
determining that at least a portion of the request cannot be fulfilled electronically by the provider; and
in response to the determination, providing the portion of the request to the provider according to the non-electronic records workflow and the criteria.

5. The method of claim 2, wherein providing the request to the provider according to the non-electronic records workflow and the criteria comprises:
scheduling an appointment with the provider to retrieve the non-electronic records;
retrieving the non-electronic records from the provider in accordance with the appointment; and
converting the non-electronic records into an electronic format according to the criteria associated with the request.

6. The method of claim 1, further comprising:
receiving from some of the providers in the subset electronic records responsive to the request by the computing device;
receiving from some of the providers in the subset non-electronic records responsive to the request by the computing device;
providing the electronic records responsive to the request to the payor by the computing device; and
providing the non-electronic records responsive to the request to the payor by the computing device.

7. The method of claim 1, wherein the criteria indicates that no non-electronic records should be retrieved, and only selecting providers for the subset that are determined to use electronic records.

8. The method of claim 1, wherein the criteria include a due date, and further comprising:
determining providers that can provide documents by the due date; and
only selecting providers for the subset that can provide documents by the due date.

9. The method of claim 1, wherein the criteria include a use for the records; and further comprising:
based on the use for the records determining whether the request can be fulfilled using only electronic records, or a combination of electronic and non-electronic records;
if it is determined that the request can be fulfilled using only electronic records:
only selecting providers for the subset that are determined to be active in the electronic records system; and
providing the request to the to the providers in the subset according to an electronic records workflow and the criteria.

10. The method of claim 9, further comprising:
if it is determined that the request can be fulfilled using a combination of electronic and non-electronic records:
selecting providers for the subset that are determined to be active in the electronic records system;
selecting providers for the subset that are determined to be not active in the electronic records system s;
providing the request to the to the providers in the subset determined to use electronic records according to an electronic records workflow and the criteria; and
providing the request to the to the providers in the subset determined to not use electronic records according to a non-electronic records workflow and the criteria.

11. The method of claim 1, wherein the records are medical records, the providers are medical providers, and the payor is an insurance payor.

12. A system comprising:
at least one processor; and
a computer readable medium with computer executable instructions stored thereon that when executed by the at least one processor causes the system to:
receive a request for records from a payor, wherein the request includes a plurality of provider identifiers and criteria and further wherein each provider identifier identifies a provider of a plurality of providers;
for each provider associated with a provider identifier from the plurality of provider identifiers, determine whether the provider is active in an electronic records system;
based on the determination of whether the providers are active in the electronic records system and the criteria, select a subset of the providers associated with a provider identifier of the plurality of provider identifiers; and
provide the request to each provider in the subset according to the criteria.

13. The system of claim 12, wherein providing the request to each provider in the subset according to the criteria comprises:
for each provider in the subset determined to be active in the electronic records system, providing the request to the provider according to an electronic records workflow and the criteria;
for each provider in the subset determined to be active in the electronic records system, providing the request to the provider according to a non-electronic records workflow and the criteria.

14. The system of claim 13, wherein providing the request to the provider according to the electronic records workflow and the criteria comprises:
providing the request electronically to the provider;
receiving from the provider electronic records responsive to the request; and
converting electronic records into a format specified by the payor in the criteria.

15. The system of claim 14, further comprising:
determining that at least a portion of the request cannot be fulfilled electronically by the provider; and
in response to the determination, providing the portion of the request to the provider according to the non-electronic records workflow and the criteria.

16. The system of claim 13, wherein providing the request to the provider according to the non-electronic records workflow and the criteria comprises:
scheduling an appointment with the provider to retrieve the non-electronic records;
retrieving the non-electronic records from the provider in accordance with the appointment; and
converting the non-electronic records into an electronic format according to the criteria associated with the request.

17. The system of claim 12, further comprising:
receiving from some of the providers in the subset electronic records responsive to the request by the computing device;
receiving from some of the providers in the subset non-electronic records responsive to the request by the computing device;
providing the electronic records responsive to the request to the payor by the computing device; and
providing the non-electronic records responsive to the request to the payor by the computing device.

18. The system of claim 12, wherein the criteria indicates that no non-electronic records should be retrieved, and only selecting providers for the subset that are determined to use electronic records.

19. The system of claim 12, wherein the criteria include a due date, and further comprising:
determining providers that can provide documents by the due date; and
only selecting providers for the subset that can provide documents by the due date.

20. A non-transitory computer readable medium with computer executable instructions stored thereon that when executed by at least one processor causes a system to:
receive a request for records from a payor, wherein the request includes a plurality of provider identifiers and criteria and further wherein each provider identifier identifies a provider of a plurality of providers;
for each provider associated with a provider identifier from the plurality of provider identifiers, determine whether the provider is active in an electronic records system;
based on the determination of whether the providers are active in the electronic records system and the criteria, select a subset of the providers associated with a provider identifier of the plurality of provider identifiers; and
provide the request to each provider in the subset according to the criteria.
